(19) United States
(12) Patent Application Publication
(10) Pub. No.: US 2023/0210842 A1
(43) Pub. Date: Jul. 6, 2023

(54) PREPARATION METHOD AND APPLICATION OF SUPRAMOLECULAR NANO-DRUG BASED ON IRINOTECAN AND NIRAPARIB

(71) Applicant: THE EIGHTH AFFILIATED HOSPITAL, SUN YAT-SEN UNIVERSITY, Shenzhen (CN)

(72) Inventors: Leilei SHI, Shenzhen (CN); Peng ZHANG, Shenzhen (CN); Yuzhen LI, Shenzhen (CN); Tong CHEN, Shenzhen (CN); Miaomiao YUAN, Shenzhen (CN)

(73) Assignee: THE EIGHTH AFFILIATED HOSPITAL, SUN YAT-SEN UNIVERSITY, Shenzhen (CN)

(21) Appl. No.: 17/973,581

(22) Filed: Oct. 26, 2022

(30) Foreign Application Priority Data
Dec. 30, 2021 (CN) 202111657002.4

(51) Int. Cl. A61K 31/4745; A61K 31/454; A61K 9/51; A61P 35/00

(52) U.S. Cl. CPC A61K 31/4745; A61K 31/454; A61K 9/51; A61P 35/00; B82Y 5/00

(57) ABSTRACT

A supramolecular nano-drug based on irinotecan and niraparib, and a preparation method and application thereof are provided. The nano-drug is a stable supramolecular nanostructure with a regular geometric appearance, which is formed by self-assembling irinotecan and niraparib through supramolecular acting forces, such as hydrogen bonds, and hydrophobic Van Der Waals force by a dynamic supramolecular self-assembly method. The supramolecular nano-drug formed in the present disclosure is capable of significantly improving the sensitivity of colorectal cells to the irinotecan, and the quantity of DNA damages induced in the colorectal cells is significantly higher than that induced by the irinotecan as a single chemotherapeutic drug. The supramolecular nano-drug of the present disclosure can not only induce death of colorectal cancer cells in vitro, but also play a role in treating the colorectal cancer in vivo after being administrated intravenously.

### CROSS REFERENCE TO THE RELATED APPLICATIONS

This application is based upon and claims priority to Chinese Patent Application No. 202111657002.4, filed on Dec. 30, 2021, the entire contents of which are incorporated herein by reference.

### TECHNICAL FIELD

The present disclosure relates to the technical field of nano-drug development, in particular to a preparation method and application of a supramolecular nano-drug based on irinotecan and niraparib.

### BACKGROUND

Irinotecan is a camptothecin semi-synthetic derivative, and is mainly used for treating advanced metastatic colorectal cancers. By inhibiting topoisomerase I essential for tumor cell DNA replication, the irinotecan induces DNA single-strand damage, and blocks DNA replication to produce cytotoxicity. Niraparib as a PARP inhibitor is capable of inhibiting repair of tumor cell DNA single-strand damage, resulting in accumulation of single-strand damages, which induces DNA double-strand break, and may achieve synthetic lethal effects on tumors having homologous recombination repair defects. The irinotecan and the niraparib affect damage and repair phases of the tumor cell DNA respectively, and the irinotecan and the niraparib are combined to have the synergistic anti-tumor activity. As the current clinical chemotherapy regimen has poorer therapeutic effect on the advanced metastatic colorectal cancers, designing and preparing a self-assembled nanomolecular drug based on the irinotecan and the niraparib will have important clinical application values.

### SUMMARY

The present invention aims to design and prepare a supramolecular polymer based on irinotecan and niraparib according to a molecular structure and a pharmacological mechanism of chemotherapeutic drugs to solve the problems that the chemotherapeutic drug irinotecan and conventional chemotherapy regimens for advanced colorectal cancers have poor therapeutic effect, easiness in drug resistance, and more adverse reactions and the like.

According to an aspect of the present disclosure, a supramolecular nano-drug for treating colorectal cancer is provided, and the supramolecular nano-drug includes irinotecan and niraparib.

In the supramolecular nano-drug provided by the present disclosure, a molar ratio of the irinotecan to the niraparib is 1:2 to 1:3.

According to another aspect of the present invention, a preparation method of the supramolecular nano-drug based on the irinotecan and the niraparib is further provided, and the preparation method includes the following steps:

S1, dissolving the irinotecan and the niraparib into dimethyl sulfoxide respectively, fully mixing the two obtained solutions, adding double distilled water to the mixed solution, slowly stirring the solution by a magnetic stirrer, and shaking the solution on a shaker after the liquid becomes clear from being turbid;

S2, ultra-filtering the solution obtained in step S1 to remove an organic solvent; and

S3, freeze-drying the ultra-filtered solution to obtain solid powder of the nano-drug.

In the preparation method provided by the present disclosure, in step S1, the solution concentration of the irinotecan and the niraparib is 10 mg/ml to 50 mg/ml; the molar ratio of the irinotecan to the niraparib is 1:2 to 1:3; a volume of the added double distilled water is 20 to 100 times that of the mixed solution of the irinotecan and the niraparib; a stirring speed of the magnetic stirrer is 100 rpm to 300 rpm, and a reaction temperature is room temperature; and the shaking time of the shaker is 1 hour to 3 hours.

In the preparation method provided by the present disclosure, in step S2, a molecular weight cut-off of an ultra-filtration centrifuge tube used in the ultra-filtering process is 10000 dalton, a rotational speed is 3000 g, and the time is 10 minutes.

According to yet another aspect of the present disclosure, an application of the supramolecular nano-drug in treatment of the colorectal cancer is further provided.

The preparation method and application of the supramolecular nano-drug based on the irinotecan and the niraparib in the present disclosure have the following beneficial effects that compared with conventional chemotherapeutic drugs, the self-assembled supramolecular nano-drug can improve its bioavailability, increase targeting property and improve stability and controlled release effect, and has better therapeutic effects. Therefore, compared with irinotecan monotherapy, the supramolecular polymer based on the irinotecan and the niraparib provided by the present disclosure obviously increases the quantity of DNA single-strand damages induced and generated in cancer cells, and significantly improves the effect of killing tumor cells; and the supramolecular polymer is easy to absorb by the tumor cells, and can be properly used for treating the colorectal cancer.

### BRIEF DESCRIPTION OF THE DRAWINGS

In order to more clearly explain the embodiments of the disclosure or the technical solutions of the prior art, the drawings needed in the description of the embodiments or the prior art will be briefly described. Obviously, the drawings in the following description are only embodiments of the present disclosure. For those ordinarily skilled in the art, other drawings can be obtained in accordance with the provided drawings without involving any creative work:

FIG. 1 illustrates molecular structures of drug irinotecan and niraparib involved in the present disclosure;

FIG. 2 illustrates DLS particle sizes of supramolecular polymer nanomaterials (IN) in the present disclosure;

FIG. 3 illustrates a picture of IN under a transmission electron microscope;

FIG. 4 illustrates influences of irinotecan, niraparib and IN on cell viability of colorectal cancer cells HCT116;

FIG. 5 illustrates influences of the irinotecan, the niraparib and the IN on cell viability of irinotecan-resistant cell lines HCT8/v;

FIG. 6 illustrates cell clones formed by growth and proliferation of the colorectal cancer cells treated by the irinotecan, the niraparib and the IN; and

FIG. 7 illustrates the observation of the quantity of cell DNA damages caused by the induction of irinotecan, the niraparib and the IN by a laser scanning confocal microscope.

### DETAILED DESCRIPTION OF THE EMBODIMENTS

In order to conveniently understand the present disclosure, the present disclosure will be described below more comprehensively with reference to the related drawings. Typical embodiments of the present disclosure are given in the drawings. However, the present disclosure may be achieved in many different forms, and is not limited to the embodiments described here. On the contrary, the purpose of providing these embodiments is to make the contents disclosed by the present disclosure clearer and more comprehensive.

Unless otherwise defined, all technical terms used here have the same meaning as commonly understood by those skilled in the art. The terms used here in the specification of the present disclosure are only used for describing the objectives of the specific embodiments, rather than limiting the present disclosure.

A preparation method of a supramolecular nano-drug based on irinotecan and niraparib in the present disclosure will be further described with reference to the drawings and the embodiments:

The preparation method of the supramolecular nano-drug based on the irinotecan and the niraparib includes the following steps:

S1, the irinotecan and the niraparib are dissolved into dimethyl sulfoxide respectively, the two obtained solutions are fully mixed, double distilled water is added to the mixed solution, the solution is slowly stirred by a magnetic stirrer, and shaken on a shaker after the liquid becomes clear from being turbid;

Specifically, in step S1, the solution concentration of the irinotecan and the niraparib is 10 mg/ml to 50 mg/ml; the molar ratio of the irinotecan to the niraparib is 1:2 to 1:3; a volume of the added double distilled water is 20 to 100 times that of the mixed solution of the irinotecan and the niraparib; a stirring speed of the magnetic stirrer is 100 rpm to 300 rpm, and a reaction temperature is room temperature; and the shaking time of the shaker is 1 hour to 3 hours.

S2, ultra-filtering the solution obtained in step S1 to remove an organic solvent; and

Specifically, a molecular weight cut-off of an ultra-filtration centrifuge tube used in the ultra-filtering process is 10000 dalton, a rotational speed is 3000 g, and the time is 10 minutes.

S3, the ultra-filtered solution is freeze-dried to obtain solid powder of the nano-drug.

The embodiments of the present disclosure will be further described in multiple embodiments. Wherein, the embodiments of the present disclosure are not limited to the following specific embodiments. Changes may be implemented appropriately without changing the scope of the claims.

### Embodiment 1

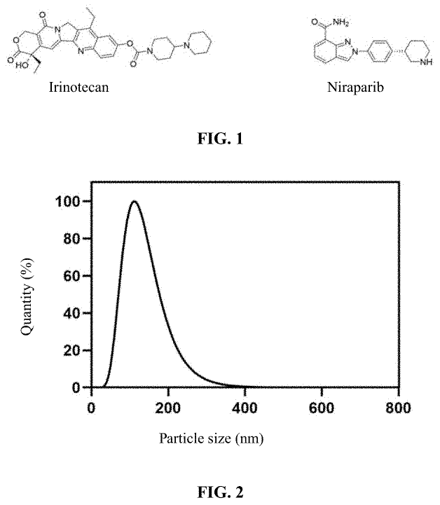

The embodiment relates to a supramolecular polymer based on irinotecan and niraparib, and a preparation method thereof is as follows: The irinotecan (10 μmol, 5.87 mg) and the niraparib (20 μmol, 6.41 mg) with structures shown in FIG. 1 were dissolved in DMSO (250 μl), and fully dissolved by vortex oscillation; after double distilled water (10 ml) was added to the obtained solution, the solution was stirred by a magnetic stirrer at 300 rpm for 10 minutes or so, and horizontally shaken on a shaker for 2 hours after the liquid became clear from being turbid; then, the liquid was ultra-filtered by an ultra-filtration centrifuge tube with a molecular weight cut-off of 10000 dalton, centrifuged under a centrifugal force of 3000 g for 5 minutes, and the ultra-filtered liquid was collected, and quickly frozen in a refrigerator at −80° C.; and then, the obtained ice was sublimated and dried by a freeze dryer to obtain solid powder of the supramolecular polymer nano-drug.

### Embodiment 2

The embodiment relates to a supramolecular polymer based on irinotecan and niraparib, and a preparation method thereof is as follows: The irinotecan (10 μmol, 5.87 mg) and the niraparib (25 μmol, 8.01 mg) with structures shown in FIG. 1 were dissolved in DMSO (250 μl), and fully dissolved by vortex oscillation; after double distilled water (10 ml) was added to the obtained solution, the solution was stirred by a magnetic stirrer at 300 rpm for 10 minutes or so, and horizontally shaken on a shaker for 2 hours after the liquid became clear from being turbid; then, the liquid was ultra-filtered by an ultra-filtration centrifuge tube with a molecular weight cut-off of 10000 dalton, centrifuged under a centrifugal force of 3000 g for 5 minutes, and the ultra-filtered liquid was collected, and quickly frozen in a refrigerator at −80° C.; and then, the obtained ice was sublimated and dried by a freeze dryer to obtain solid powder of the supramolecular polymer nano-drug.

### Embodiment 3

The embodiment relates to a supramolecular polymer based on irinotecan and niraparib, and a preparation method thereof is as follows: The irinotecan (10 μmol, 5.87 mg) and the niraparib (30 μmol, 9.61 mg) with structures shown in FIG. 1 were dissolved in DMSO (250 μl), and fully dissolved by vortex oscillation; after double distilled water (10 ml) was added to the obtained solution, the solution was stirred by a magnetic stirrer at 300 rpm for 10 minutes or so, and horizontally shaken on a shaker for 2 hours after the liquid became clear from being turbid; then, the liquid was ultra-filtered by an ultra-filtration centrifuge tube with a molecular weight cut-off of 10000 dalton, centrifuged under a centrifugal force of 3000 g for 5 minutes, and the ultra-filtered liquid was collected, and quickly frozen in a refrigerator at −80° C.; and then, the obtained ice was sublimated and dried by a freeze dryer to obtain solid powder of the supramolecular polymer nano-drug.

### Embodiment 4

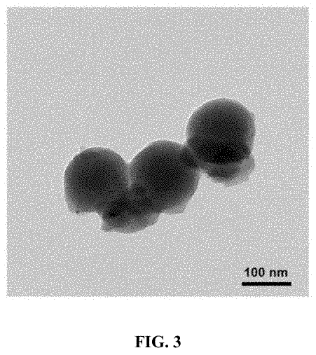

A DMSO stock solution of IN at a concentration of 1 mg/mL was prepared, the IN was diluted with double distilled water to obtain a solution with a concentration of 10 μg/mL, and an absorption spectrum of the obtained solution was determined by a Thermo Electron-EV300 ultraviolet and visible spectrophotometer at the maximum absorption wavelength of 306 nm. Then, a particle size of the IN was determined by a nanoparticle size analyzer, and the particle size of the IN was about 130 nm. Next, the morphology of the IN was observed by a transmission electron microscope, the particle size was measured and evaluated, and the IN showed a spherical structure with a diameter of 120 nm or so. FIG. 2 illustrates DLS particle sizes of supramolecular polymer nanomaterials (IN) in the present disclosure. FIG. 3 illustrates a picture of the IN under a transmission electron microscope.

### Embodiment 5

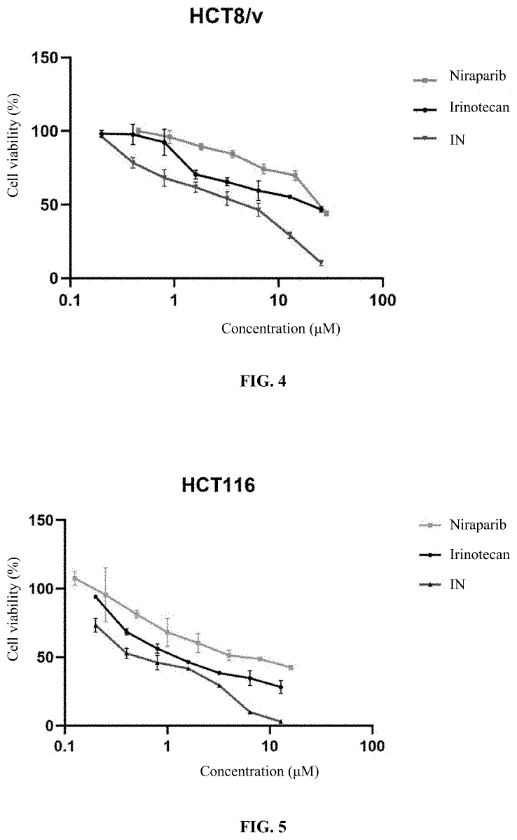

Human colorectal cancer cells HCT116 were implanted in a 96-well plate at 5000 cells per well; after the cells were adhered to the surface of the plate, irinotecan, niraparib and IN at different concentrations (200 nM, 400 nM, 800 nM, 1.6 μM, 3.2 μM, 6.4 μM, 12.8 μM and 25.6 μM) were added respectively, and the cells were continuously cultured for 72 hours; then, a mixed solution of 10 μL of CCK-8 and 100 μl of DMEM was added to each well; and after incubation was conducted in an incubator at 37° C. for 1 hour, the absorbance of each well was detected by an ELISA analyzer at a wavelength of 450 nm. Irinotecan-resistant cell lines HCT8/v were also tested as described above. FIG. 4 illustrates influences of the irinotecan, the niraparib and IN on the cell viability of the colorectal cancer cells HCT116. FIG. 5 illustrates influences of the irinotecan, the niraparib and the IN on the cell viability of irinotecan-resistant cell lines HCT8/v. The results show that the inhibiting effect of the IN on the cell viability of the colorectal cancer cells is significantly higher than that of the irinotecan and the niraparib.

### Embodiment 6

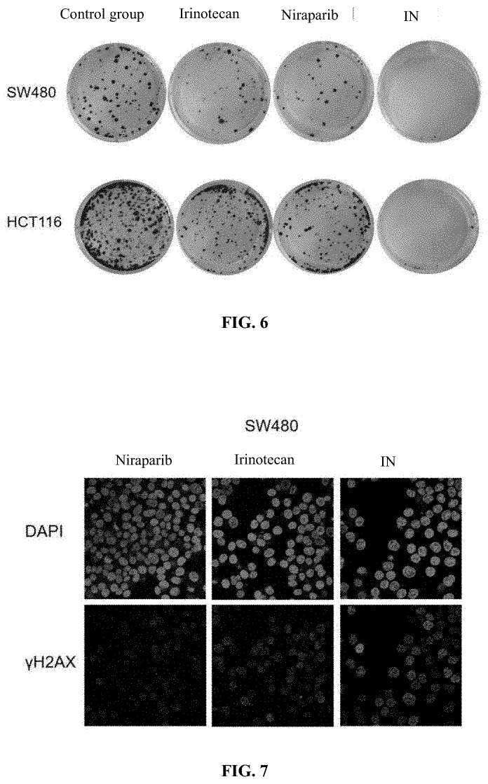

Colorectal cancer cells HCT116 were implanted in a 6-well plate at 1000 cells per well; after the cells were adhered to the surface of the plate, 0.5 μM of irinotecan, niraparib and IN were added respectively, and a culture medium was changed after the cells were cultured for 72 hours; after the cells were continuously cultured for 10 days or so, clones visible to naked eyes could be observed in the 6-well plate, and the culture was terminated; supernatants were discarded, and the cells were stained with crystal violet after being immobilized with 4% paraformaldehyde; and the cloning numbers of the cells in all groups were compared. Colorectal cancer cells SW480 were also tested as described above. FIG. 6 illustrates the cell clones formed by growth and proliferation of the colorectal cancer cells treated by the irinotecan, the niraparib and the IN. The results show that the killing effect of the IN on the colorectal cancer cells is significantly higher than that of the irinotecan and the niraparib.

### Embodiment 7

Colorectal cancer cells SW480 were implanted in confocal dishes at 104 cells per dish; and after the cells were adhered to the surface of the dish, irinotecan, niraparib and IN (0.2 μM) were added respectively. A culture medium was removed after 24 hours, and the cells were immobilized with 4% paraformaldehyde for 30 minutes; after permeabilization was implemented with 0.3% Triton X-100 for 5 minutes, gamma h2ax primary antibodies were incubated overnight, and then anti-rabbit secondary antibodies were incubated for 30 minutes; and after cell nuclei were stained with 4′,6-diamidino-2-phenylindole (DAPI), the number of gamma h2ax focuses of the cells SW480 was observed under a laser scanning confocal microscope. FIG. 7 illustrates the observation of the quantity of cell DNA damages caused by the induction of irinotecan, the niraparib and the IN by a laser scanning confocal microscope. The results show that the quantity of DNA damages induced by the IN is significantly higher than that of the irinotecan and the niraparib.

Although the embodiments of the present disclosure are described with reference to the drawings above, the present disclosure is not limited to the above specific implementations, and the above specific implementations are only exemplary instead of restrictive; and those ordinarily skilled in the art may also make many forms without departing from the purpose of the present disclosure and the scope protected by claims under inspiration of the present disclosure, and these forms all belong to the protection scope of the present disclosure.

What is claimed is:

1. A supramolecular nano-drug for treating a colorectal cancer, wherein the supramolecular nano-drug comprises irinotecan and niraparib.

2. The supramolecular nano-drug according to claim 1, wherein a molar ratio of the irinotecan to the niraparib is 1:2 to 1:3.

3. A method of preparing a supramolecular nano-drug based on irinotecan and niraparib, wherein the method comprises:
S1, dissolving the irinotecan and the niraparib into dimethyl sulfoxide respectively to obtain two solutions, fully mixing the two solutions to obtain a mixed solution, adding double distilled water to the mixed solution, stirring the mixed solution by a magnetic stirrer, and shaking the mixed solution on a shaker after a liquid becomes clear from being turbid;
S2, ultra-filtering the mixed solution obtained in step S1 to remove an organic solvent to obtain an ultra-filtered solution; and
S3, freeze-drying the ultra-filtered solution to obtain a solid powder of the supramolecular nano-drug.

4. The method according to claim 3, wherein in the step S1, a concentration of each of the two solutions is 10 mg/ml to 50 mg/ml; a molar ratio of the irinotecan to the niraparib is 1:2 to 1:3; a volume of the double distilled water is 20 to 100 times a volume of the mixed solution; a stirring speed of the magnetic stirrer is 100 rpm to 300 rpm, and a reaction temperature is a room temperature; and a shaking time of the shaker is 1 hour to 3 hours.

5. The method according to claim 3, wherein in the step S2, a molecular weight cut-off of an ultra-filtration centrifuge tube used in the ultra-filtering is 10000 dalton, a rotational speed is 3000 g, and a time of the ultra-filtering is 10 minutes.

6. A method of an application of the supramolecular nano-drug according to claim 1 in a treatment of the colorectal cancer.

7. The method according to claim 6, wherein a molar ratio of the irinotecan to the niraparib is 1:2 to 1:3.
